(19) United States
(12) Patent Application Publication
(10) Pub. No.: US 2013/0158659 A1
(43) Pub. Date: Jun. 20, 2013

(54) Medical Devices, Apparatuses, Systems, and Methods With Configurations for Shaping Magnetic-Fields and Interactions

(71) Applicants: Richard A. Bergs, Grand Prairie, TX (US); Heather E. Beardsley, Arlington, TX (US); Jeffrey A. Cadeddu, Dallas, TX (US); Raul Fernandez, Arlington, TX (US); Daniel J. Scott, Dallas, TX (US)

(72) Inventors: Richard A. Bergs, Grand Prairie, TX (US); Heather E. Beardsley, Arlington, TX (US); Jeffrey A. Cadeddu, Dallas, TX (US); Raul Fernandez, Arlington, TX (US); Daniel J. Scott, Dallas, TX (US)

(21) Appl. No.: 13/331,453

(22) Filed: Dec. 20, 2011

(51) Int. Cl. A61F 2/02

(52) U.S. Cl. 623/11.11

(57) ABSTRACT

Embodiments of apparatuses and/or medical devices, and systems and methods including apparatuses and/or medical devices, comprising one or more elements configured to define a U-shaped magnetic flux path and/or magnetic field.

### BACKGROUND

1. Field of the Invention

The present invention relates generally to medical devices, apparatuses, systems, and methods, and, more particularly, but not by way of limitation, to medical devices, apparatuses, systems, and methods for performing medical procedures at least partially within a body cavity of a patient.

2. Description of Related Art

For illustration, the background is described with respect to medical procedures (e.g., surgical procedures), which can include laparoscopy, transmural surgery, and endoluminal surgery, including, for example, natural orifice transluminal endoscopic surgery (NOTES), single-incision laparoscopic surgery (SILS), and single-port laparoscopy (SLP).

Compared with open surgery, laparoscopy can result in significantly less pain, faster convalescence and less morbidity. NOTES, which can be an even less-invasive surgical approach, may achieve similar results. However, issues such as eye-hand dissociation, a two-dimensional field-of-view, instrumentation with limited degrees of freedom, and demanding dexterity requirements can pose challenges for many laparoscopic and endoscopic procedures. One limitation of laparoscopy can be the fixed working envelope surrounding each trocar. As a result, multiple ports may be used to accommodate changes in position of the instruments or laparoscope, for example, to improve visibility and efficiency. However, the placement of additional working ports may contribute to post-operative pain and increases risks, such as additional bleeding and adjacent organ damage.

The following published patent applications include information that may be useful in understanding the present medical devices, systems, and methods, and each is incorporated by reference in its entirety: (1) International Application No. PCT/US2009/063987, filed on Nov. 11, 2009, and published as WO 2010/056716; (2) U.S. patent application Ser. No. 10/024,636, filed Dec. 14, 2001, and published as Pub. No. US 2003/0114731; (3) U.S. patent application Ser. No. 10/999,396, filed Nov. 30, 2004, published as Pub. No. US 2005/0165449, and issued as U.S. Pat. No. 7,429,259; (4) U.S. patent application Ser. No. 11/741,731, filed Apr. 28, 2007, published as Pub. No. US 2007/0255273 and issued as U.S. Pat. No. 7,691,103; (5) U.S. patent application Ser. No. 12/146,953, filed Jun. 26, 2008, and published as Pub. No. US 2008/0269779; (6) International Patent Application No. PCT/US10/21292, filed Jan. 16, 2010, and published as WO 2010/083480.

### SUMMARY

This disclosure includes embodiments of medical devices, apparatuses, platforms, systems, and methods.

Some embodiments of the present medical devices comprise: a platform configured to be inserted within a body cavity of a patient (e.g., where the platform comprises: three or more elements each comprising at least one of a magnetically attractive and magnetically-chargeable material, the three elements at least partially defining a U-shaped magnetic flux path). In some embodiments, the one or more elements comprise: a first element comprising at least one of a magnetically-attractive material and magnetically-chargeable material, the first element having a first magnetic orientation; a second element comprising at least one of a magnetically-attractive material and magnetically-chargeable material, the second element having a second magnetic orientation; and a third element comprising at least one of a magnetically-attractive material and magnetically-chargeable material; where the second element is spaced apart from the first element, the second magnetic orientation is opposite the first magnetic orientation, and the third element extends between the first element and the second element. In some embodiments, the third element has a third magnetic orientation independent of the first and second elements. In some embodiments, the third magnetic orientation is substantially perpendicular to the first and second magnetic orientations. In some embodiments, the third element has an elongated shape and a central longitudinal axis. In some embodiments, the third element has a first mating surface at a first end, and a second mating surface at a second end. In some embodiments, the first and second mating surfaces of the third element are substantially perpendicular to the longitudinal axis. In some embodiments, the first and second mating surfaces of the third element are disposed at non-perpendicular angles relative to the longitudinal axis. In some embodiments, the non-perpendicular angles are between 40 and 50 degrees. In some embodiments, the first and second elements have substantially identical cross-sectional shapes. In some embodiments, the first, second, and third elements have substantially identical cross-sectional shapes.

Some embodiments of the present apparatuses comprise: a platform configured to be magnetically coupled to a medical device disposed within a body cavity of a patient through a tissue (e.g., where the platform comprises: a body; and three elements each comprising at least one of a magnetically attractive and magnetically-chargeable material, the three elements at least partially defining a U-shaped magnetic flux path). In some embodiments, the one or more elements comprise: a first element comprising at least one of a magnetically-attractive material and magnetically-chargeable material, the first element having a first magnetic orientation; a second element comprising at least one of a magnetically-attractive material and magnetically-chargeable material, the second element having a second magnetic orientation; and a third element comprising at least one of a magnetically-attractive material and magnetically-chargeable material; where the second element is spaced apart from the first element, the second magnetic orientation is opposite the first magnetic orientation, and the third element extends between the first element and the second element. In some embodiments, the third element has a third magnetic orientation independent of the first and second elements. In some embodiments, the third magnetic orientation is substantially perpendicular to the first and second magnetic orientations. In some embodiments, the third element has an elongated shape and a central longitudinal axis. In some embodiments, the third element has a first mating surface at a first end, and a second mating surface at a second end. In some embodiments, the first and second mating surfaces of the third element are substantially perpendicular to the longitudinal axis. In some embodiments, the first and second mating surfaces of the third element are disposed at non-perpendicular angles relative to the longitudinal axis. In some embodiments, the non-perpendicular angles are between 40 and 50 degrees. In some embodiments, the first and second elements have substantially identical cross-sectional shapes. In some embodiments, the first, second, and third elements have substantially identical cross-sectional shapes.

Some embodiments of the present systems comprise: any of the present apparatuses; and a medical device configured to be inserted within a body cavity of a patient (e.g., where the medical device comprises: a platform comprising one or more elements having at least one of a magnetically attractive and magnetically-chargeable material). In some embodiments, the one or more elements of the medical device at least partially define a U-shaped magnetic flux path. In some embodiments, the apparatus is magnetically coupled to the medical device. In some embodiments, the one or more elements of the medical device comprise: a first element comprising at least one of a magnetically-attractive material and magnetically-chargeable material, the first element having a first magnetic orientation; a second element comprising at least one of a magnetically-attractive material and magnetically-chargeable material, the second element having a second magnetic orientation; and a third element comprising at least one of a magnetically-attractive material and magnetically-chargeable material; where the second element is spaced apart from the first element, the second magnetic orientation is opposite the first magnetic orientation, and the third element extends between the first element and the second element. In some embodiments, the apparatus is magnetically coupled to the medical device.

Some embodiments of the present systems comprise: an apparatus configured to be coupled to a medical device within a body cavity of a patient; and any of the present medical devices. In some embodiments, the apparatus is magnetically coupled to the medical device.

Any embodiment of any of the present medical devices, apparatuses, platforms, systems, and methods can consist of or consist essentially of—rather than comprise/include/contain/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

Details associated with the embodiments described above and others are presented below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the embodiment depicted in the figures.

FIG. 1 depicts a graphical representation of one of the present medical devices positioned within a body cavity of a patient and magnetically coupled to a positioning apparatus that is located outside the cavity.

FIG. 2 is an end view of the medical device and positioning apparatus shown in FIG. 1.

FIGS. 3A-3B depict a bottom view and a side cross-sectional view, respectively, respectively, of an embodiment of the positioning apparatus shown in FIG. 1.

FIG. 4 depicts a perspective view of two elements for the present medical devices.

FIG. 5 depicts a perspective view of an embodiment of three elements for the present medical devices.

FIG. 6 depicts a side view of the embodiment of FIG. 5.

FIG. 7 depicts a perspective view of a second embodiment of three elements for the present medical devices.

FIGS. 8A and 8B depict side and end views, respectively, of the embodiment of FIG. 7.

FIG. 9 depicts a perspective view of one of the present systems that includes a third embodiment of three elements for the present medical device (medical-device embodiment) and an embodiment of three elements for the present apparatuses (apparatus embodiment).

FIGS. 10A and 10B depict end and side views, respectively, of the third medical-device embodiment of FIG. 9.

FIG. 11 depicts a side view of the first apparatus embodiment of FIG. 9.

FIG. 12 depicts a side view of a second embodiment of three elements for the present apparatuses.

### DESCRIPTION OF ILLUSTRATIVE EMBODIMENTS

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a device or kit that “comprises,” “has,” “includes” or “contains” one or more elements possesses those one or more elements, but is not limited to possessing only those elements. Likewise, a method that “comprises,” “has,” “includes” or “contains” one or more steps possesses those one or more steps, but is not limited to possessing only those one or more steps.

Further, a device or system that is configured in a certain way is configured in at least that way, but it can also be configured in other ways than those specifically described.

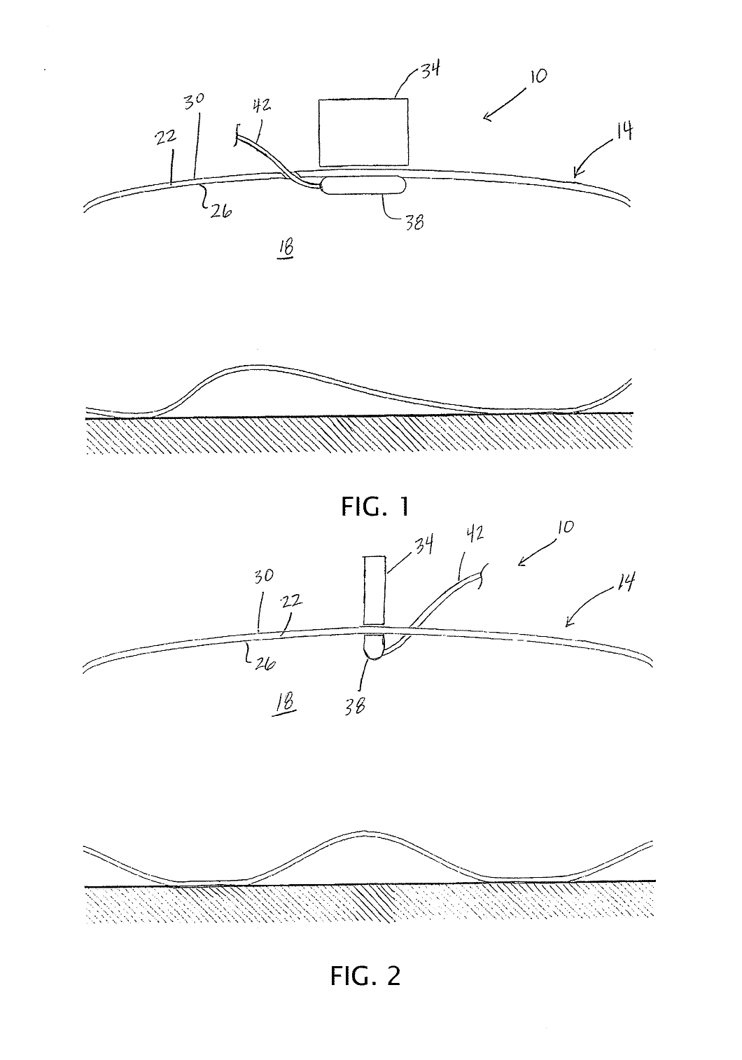

Referring now to the drawings, shown in FIGS. 1 and 2 by reference numeral 10 is one embodiment of a system for medical procedures that can be used with the present invention. System 10 is shown in conjunction with a patient 14, and more particularly in FIG. 1 is shown relative to a longitudinal cross-sectional view of the ventral cavity 18 of a human patient 14, and in FIG. 2 is shown relative to a transverse cross-sectional view of the ventral cavity of the patient. For brevity, cavity 18 is shown in simplified conceptual form without organs and the like. Cavity 18 is at least partially defined by wall 22, such as the abdominal wall, that includes an interior surface 26 and an exterior surface 30. The exterior surface 30 of wall 22 can also be an exterior surface 30 of the patient 14. Although patient 14 is shown as human in FIGS. 1 and 2, various embodiments of the present invention (including the version of system 10 shown in FIGS. 1 and 2) can also be used with other animals, such as in veterinary medical procedures.

Further, although system 10 is depicted relative to ventral cavity 18, system 10 and various other embodiments of the present invention can be utilized in other body cavities of a patient, human or animal, such as, for example, the thoracic cavity, the abdominopelvic cavity, the abdominal cavity, the pelvic cavity, and other cavities (e.g., lumens of organs such as the stomach, colon, or bladder of a patient). In some embodiments of the present methods, and when using embodiments of the present devices and systems, a pneumoperitoneum may be created in the cavity of interest to yield a relatively-open space within the cavity.

As shown in FIGS. 1 and 2, system 10 comprises an apparatus 34 and a medical device 38; the apparatus is configured to magnetically position the device with a body cavity of a patient. In some embodiments, apparatus 34 can be described as an exterior apparatus and/or external unit and device 38 as an interior device and/or internal unit due the locations of their intended uses relative to patients. As shown, apparatus 34 can be positioned outside the cavity 18 near, adjacent to, and/or in contact with the exterior surface 30 of the patent 14. Device 38 is positionable (can be positioned), and is shown positioned, within the cavity 18 of the patient 14 and near, adjacent to, and/or in contact with the interior surface 26 of wall 22. Device 38 can be inserted or introduced into the cavity 18 in any suitable fashion. For example, the device 18 can be inserted into the cavity through a puncture (not shown) in wall 22, through a tube or trocar (not shown) extending into the cavity 18 through a puncture or natural orifice (not shown), or may be inserted into another portion of the patient 14 and moved into the cavity 18 with apparatus 34, such as by the methods described in this disclosure. If the cavity 18 is pressurized, device 38 can be inserted or introduced into the cavity 18 before or after the cavity 18 is pressurized.

Additionally, some embodiments of system 10 include a version of device 38 that has a tether 42 coupled to and extending away from the device 38. In the depicted embodiment, tether 42 extends from device 38 and out of the cavity 18, for example, through the opening (not shown) through which device 38 is introduced into the cavity 18. The tether 42 can be flexible and/or elongated. In some embodiments, the tether 42 can include one or more conduits for fluids that can be used, for example, for actuating a hydraulic cylinder or irrigating a region within the cavity 18. In some embodiments, the tether 42 can include one or more conductors for enabling electrical communication with the device 38. In some embodiments, the tether 42 can include one or more conduits for fluid and one or more conductors. In some embodiments, the tether does not include a conduit or conductor and, instead, includes a cord for positioning, moving, or removing device 38 from the cavity 18. The tether 14, for example, can be used to assist in positioning the device 34 while the device 34 is magnetically coupled to the apparatus 38, or to remove the device 34 from the cavity 18 when device 38 is not magnetically coupled to apparatus 34.

As is discussed in more detail below, apparatus 34 and device 38 can be configured to be magnetically couplable to one another such that device 38 can be positioned or moved within the cavity 18 by positioning or moving apparatus 34 outside the cavity 18. “Magnetically couplable” means capable of magnetically interacting so as to achieve a physical result without a direct physical connection. Examples of physical results are causing device 38 to move within the cavity 18 by moving apparatus 34 outside the cavity 18, and causing device 38 to remain in a position within the cavity 18 or in contact with the interior surface 26 of wall 22 by holding apparatus 34 in a corresponding position outside the cavity 18 or in contact with the exterior surface 30 of wall 22. Magnetic coupling can be achieved by configuring apparatus 34 and device 38 to cause a sufficient magnetic attractive force between them. For example, apparatus 34 can comprise one or more magnets (e.g., permanent magnets, electromagnets, or the like) and device 38 can comprise a ferromagnetic material. In some embodiments, apparatus 34 can comprise one or more magnets, and device 38 can comprise a ferromagnetic material, such that apparatus 34 attracts device 38 and device 38 is attracted to apparatus 34. In other embodiments, both apparatus 34 and device 38 can comprise one or more magnets such that apparatus 34 and device 38 attract each other.

The configuration of apparatus 34 and device 38 to cause a sufficient magnetic attractive force between them can be a configuration that results in a magnetic attractive force that is large or strong enough to compensate for a variety of other factors (such as the thickness of any tissue between them) or forces that may impede a desired physical result or desired function. For example, when apparatus 34 and device 38 are magnetically coupled as shown, with each contacting a respective surface 26 or 30 of wall 22, the magnetic force between them can compress wall 22 to some degree such that wall 22 exerts a spring or expansive force against apparatus 34 and device 38, and such that any movement of apparatus 34 and device 38 requires an adjacent portion of wall 22 to be similarly compressed. Apparatus 34 and device 38 can be configured to overcome such an impeding force to the movement of device 38 with apparatus 34. Another force that the magnetic attractive force between the two may have to overcome is any friction that exists between either and the surface, if any, that it contacts during a procedure (such as apparatus 34 contacting a patient's skin). Another force that the magnetic attractive force between the two may have to overcome is the force associated with the weight and/or tension of the tether 42 and/or frictional forces on the tether 42 that may resist, impede, or affect movement or positioning of device 38 using apparatus 34.

In some embodiments, device 38 can be inserted into cavity 18 through an access port having a suitable internal diameter. Such access ports includes those created using a conventional laparoscopic trocar, gel ports, those created by incision (e.g., abdominal incision), and natural orifices. Device 38 can be pushed through the access port with any elongated instrument such as, for example, a surgical instrument such as a laparoscopic grasper or a flexible endoscope.

In embodiments where the tether 42 is connectable to a power source or a hydraulic source (not shown), the tether can be connected to the power source or the hydraulic source (which may also be described as a fluid source) either before or after it is connected to device 38.

In some embodiments, when device 38 is disposed within cavity 18, device 38 can be magnetically coupled to apparatus 34. This can serve several purposes including, for example, to permit a user to move device 38 within cavity 18 by moving apparatus 34 outside cavity 18. The magnetic coupling between the two can be affected by a number of factors, including the distance between them. For example, the magnetic attractive force between device 38 and apparatus 34 increases as the distance between them decreases. As a result, in some embodiments, the magnetic coupling can be facilitated by temporarily compressing the tissue (e.g., the abdominal wall) separating them. For example, after device 38 has been inserted into cavity 18, a user (such as a surgeon) can push down on apparatus 34 (and wall 22) and into cavity 18 until apparatus 34 and device 38 magnetically couple.

In FIGS. 1 and 2, apparatus 34 and device 38 are shown at a coupling distance from one another and magnetically coupled to one another such that device 38 can be moved within the cavity 18 by moving apparatus 34 outside the outside wall 22. The “coupling distance” between two structures (e.g., apparatus 34 and device 38) is defined as a distance between the closest portions of the structures at which the magnetic attractive force between them is great enough to permit them to function as desired for a given application.

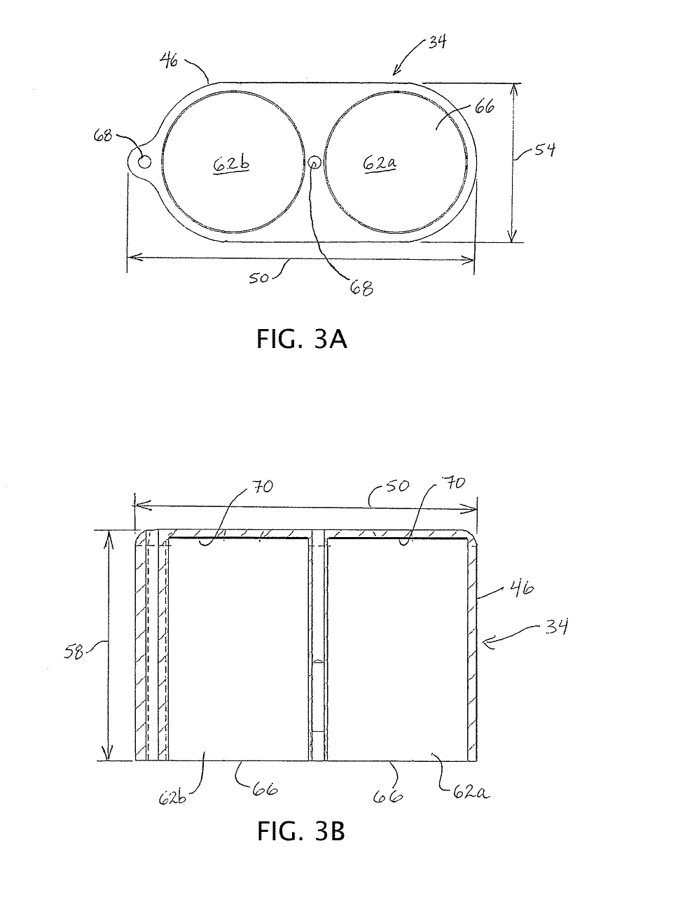

Referring now to FIGS. 3A and 3B, a bottom view and a side cross-sectional view are shown, respectively, of an embodiment of apparatus 34. Apparatus 34 has a width 50, a depth 54, and a height 58, and includes a housing 46. The apparatus (and, more specifically, housing 46) is configured to support, directly or indirectly, at least one magnetic assembly in the form of one or more magnetic field sources. In the embodiments shown, apparatus 34 is shown as including a first magnetic field source 62a and a second magnetic field source 62b. Each magnetic field source 62a, 62b has a coupling end 66 and a distal end 70. As described in more detail below, the coupling ends face device 38 when apparatus 34 and device 38 are magnetically coupled. The depicted embodiment of housing 46 of apparatus 34 also includes a pair of guide holes 68 extending through housing 46 for guiding, holding, or supporting various other devices or apparatuses, as described in more detail below. In other embodiments, the housing of apparatus 34 can have any other suitable number of guide holes 68 such as, for example, zero, one, three, four, five, or more guide holes 68. In some embodiments, housing 46 comprises a material that is minimally reactive to a magnetic field such as, for example, plastic, polymer, fiberglass, or the like. In other embodiments, housing 46 can be omitted or can be integral with the magnetic field sources such that the apparatus is, itself, a magnetic assembly comprising a magnetic field source.

Magnets, in general, have a north pole (the N pole) and a south pole (the S pole). In some embodiments, apparatus 34 can be configured (and, more specifically, its magnetic field sources can be configured) such that the coupling end 66 of each magnetic field source is the N pole and the distal end 70 of each magnetic field source is the S pole. In other embodiments, the magnetic field sources can be configured such that the coupling end 66 of each magnetic field source is the S pole and the distal end 70 of each magnetic field source is the N pole. In other embodiments, the magnetic field sources can be configured such that the coupling end of the first magnetic field source 62a is the N pole and the recessed end of the first magnetic field source 62a is the S pole, and the coupling end of the second magnetic field source 62b is the S pole and the recessed end of the second magnetic field source 62b is the N pole. In other embodiments, the magnetic field sources can be configured such that the coupling end of the first magnetic field source 62a is the S pole and its recessed end is the N pole, and the coupling end of the second magnetic field source 62b is the N pole and its recessed end is the S pole.

In the embodiment shown, each magnetic field source includes a solid cylindrical magnet having a circular cross section. In other embodiments, each magnetic field source can have any suitable cross-sectional shape such as, for example, rectangular, square, triangular, fanciful, or the like. In some embodiments, each magnetic field source comprises any of: any suitable number of magnets such as, for example, one, two, three, four, five, six, seven, eight, nine, ten, or more magnets; any suitable number of electromagnets such as, for example, one, two, three, four, five, six, seven, eight, nine, ten or more electromagnets; any suitable number of pieces of ferromagnetic material such as, for example, one, two, three, four, five, six, seven, eight, nine, ten or more pieces of ferromagnetic material; any suitable number of pieces of paramagnetic material such as, for example, one, two, three, four, five, six, seven, eight, nine, ten or more pieces of paramagnetic material; or any suitable combination of magnets, electromagnets, pieces of ferromagnetic material, and/or pieces of paramagnetic material.

In some embodiments, each magnetic field source can include four cylindrical magnets (not shown) positioned in end-to-end in linear relation to one another, with each magnet having a height of about 0.5 inch and a circular cross-section that has a diameter of about 1 inch. In these embodiments, the magnets can be arranged such that the N pole of each magnet faces the S pole of the next adjacent magnet such that the magnets are attracted to one another and not repulsed.

Examples of suitable magnets can include: flexible magnets; Ferrite, such as can comprise Barium or Strontium; AlNiCo, such as can comprise Aluminum, Nickel, and Cobalt; SmCo, such as can comprise Samarium and Cobalt and may be referred to as rare-earth magnets; and NdFeB, such as can comprise Neodymium, Iron, and Boron. In some embodiments, it can be desirable to use magnets of a specified grade, for example, grade 40, grade 50, or the like. Such suitable magnets are currently available from a number of suppliers, for example, Magnet Sales & Manufacturing Inc., 11248 Playa Court, Culver City, Calif. 90230 USA; Amazing Magnets, 3943 Irvine Blvd. #92, Irvine, Calif. 92602; and K & J Magnetics Inc., 2110 Ashton Dr. Suite 1A, Jamison, Pa. 18929. In some embodiments, one or more magnetic field sources can comprise ferrous materials (e.g., steel) and/or paramagnetic materials (e.g., aluminum, manganese, platinum).

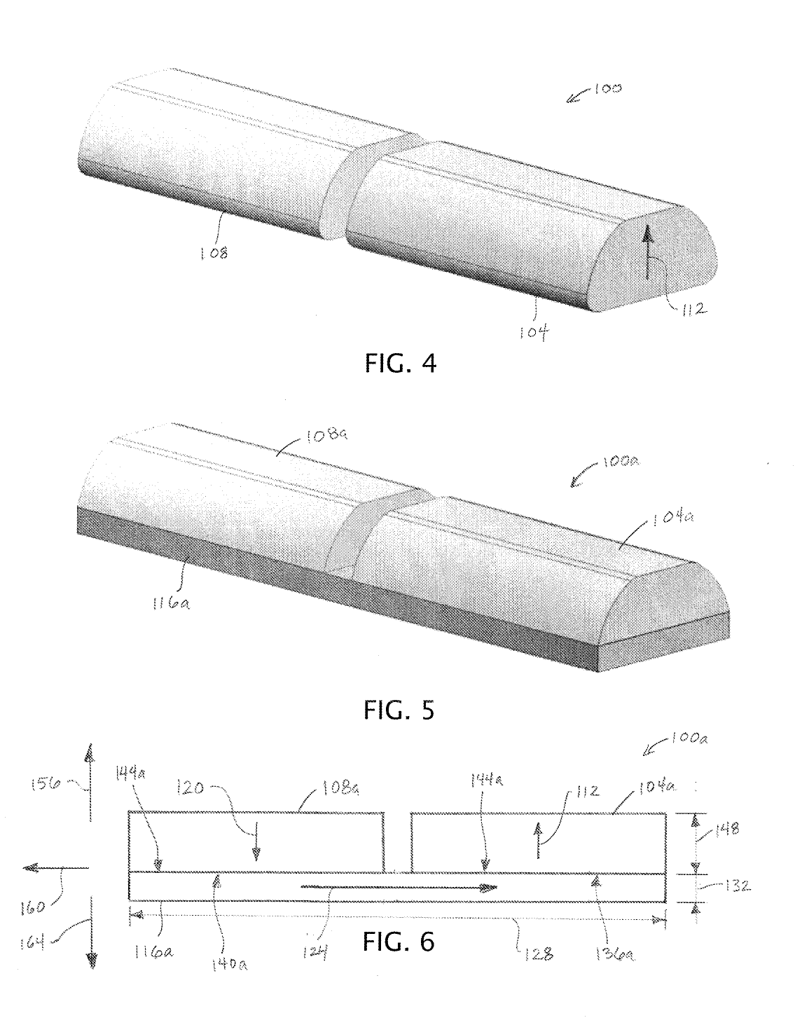

FIG. 4 depicts a perspective view of two elements for the present medical devices. In the embodiment shown, a platform 100 of a medical device (e.g., 38) can comprise: a first element 104 and a second element 108 each comprising at least one of a magnetically-attractive and a magnetically-chargeable material. Examples of magnetically-attractive and/or magnetically-chargeable materials include magnets (e.g., permanent magnets), ferrous materials (e.g., steel), and paramagnetic materials (e.g., aluminum, manganese, platinum). In the embodiment shown, first and second elements 104 and 108 each comprises a single magnet. In the embodiment shown, first and second elements 104 and 108 have substantially constant and substantially identical cross-sectional shapes along their respective longitudinal axes (which are also substantially collinear). In the embodiment shown, first element 104 is magnetized in a first direction 112, and second element 108 is magnetized in a second direction that is substantially opposite to direction 112. First and second elements 104 and 108, for example, can be similar in materials and/or function to magnetic field sources 62a and 62b, described above.

FIG. 5 depicts a perspective view of an embodiment of three elements for the present medical devices, and FIG. 6 depicts a side view of the embodiment of FIG. 5. In the embodiment shown, a platform or chassis 100a is shown for inclusion in a medical device (e.g., 38) configured to be inserted within a body cavity of a patient. Some embodiments of the present platforms include three or more elements each comprising at least one of a magnetically attractive and magnetically-chargeable material, the three elements at least partially defining a U-shaped magnetic flux path and/or magnetic field. For example, in the embodiment shown, the three or more elements comprise a first element 104a, a second element 108a, and a third element 116a, each comprising at least one of a magnetically-attractive material and magnetically-chargeable material. In this embodiment, first element 104a has a first magnetic orientation in which the first element is magnetized and/or magnetizable in a first direction 112; and second element 108a has a second magnetic orientation in which the second element is magnetized and/or magnetizable in a direction 120 that is substantially opposite direction 112. In the embodiment shown, second element 108a is spaced apart from first element 104a, and third element 116a extends between the first element and the second element.

In the embodiment shown, first and second elements 104a and 108a each comprises a magnet magnetized in an N-S direction 112 or 120, respectively. In other embodiments, each of the first and second elements can include a plurality of magnets. In the embodiment shown, third element 116a comprises a ferrous material (e.g., steel such as, for example, a mild steel) that need not be magnetized prior to being in proximity to the first and second elements. In other embodiments, third element 116a can comprise multiple pieces of material. In the embodiment shown, third element 116a has a third magnetic orientation in which the third element is magnetized and/or magnetizable in direction 124 that is substantially perpendicular to both of directions 112 and 120. In this embodiment, the magnetic orientation of third element 116a is dependent on the first and second magnetic orientations of the first and second elements, respectively. However, in other embodiments, third element 116a can comprise a magnet such that a magnetic orientation in which the third element is magnetized in direction 124 would exist independently of the magnetic orientations of first and second elements 104a and 108a. In the embodiment shown, third element 116a has an elongated shape in which a length 128 of the third element is larger (e.g., 200%, 500%, 1000%, or more) than a height or thickness 132 of the third element. In the embodiment shown, bottom mating surfaces 136a and 140a of first and second elements 104a and 108a, respectively, contact or mate with a top mating surface 144a of the third element. In the embodiment shown, height or thickness 132 is less than (e.g., equal to, less than, or between any of: 70%, 60%, 50%, 40%, 30% of) height or thickness 148 of first element 104a (and second element 108a). In some embodiments, height or thickness 132 of the third element can be 0.070 inches, and height or thickness 148 of first element 104a can be 0.0156 inches. As such, in the embodiment shown, height 132 is about 31% of the overall height (sum of heights 132 and 148) and height 148 is about 69% of the overall height. In other embodiments, height 132 can be between 20% and 40% (e.g., between 25% and 35%) of the overall height, and height 148 can be between 80% and 60% (e.g., between 75% and 65%) of the overall height. In the embodiment shown, the length of each of elements 104a and 104b (parallel to length 128) is 1.85 inches. In the embodiment shown, the inclusion of third element 116a increases the magnetic force in upward direction 156, and reduces the overall magnetic field projection in outward direction 160 and downward direction 164, relative to a configuration (FIG. 4) without the third element (with only the first and second elements). In various embodiments, platform 100a (and/or platforms 100b and 100c, described below) can include one or more tools, such as, for example, one or more of a camera, a light, a cautery, and/or other tools.

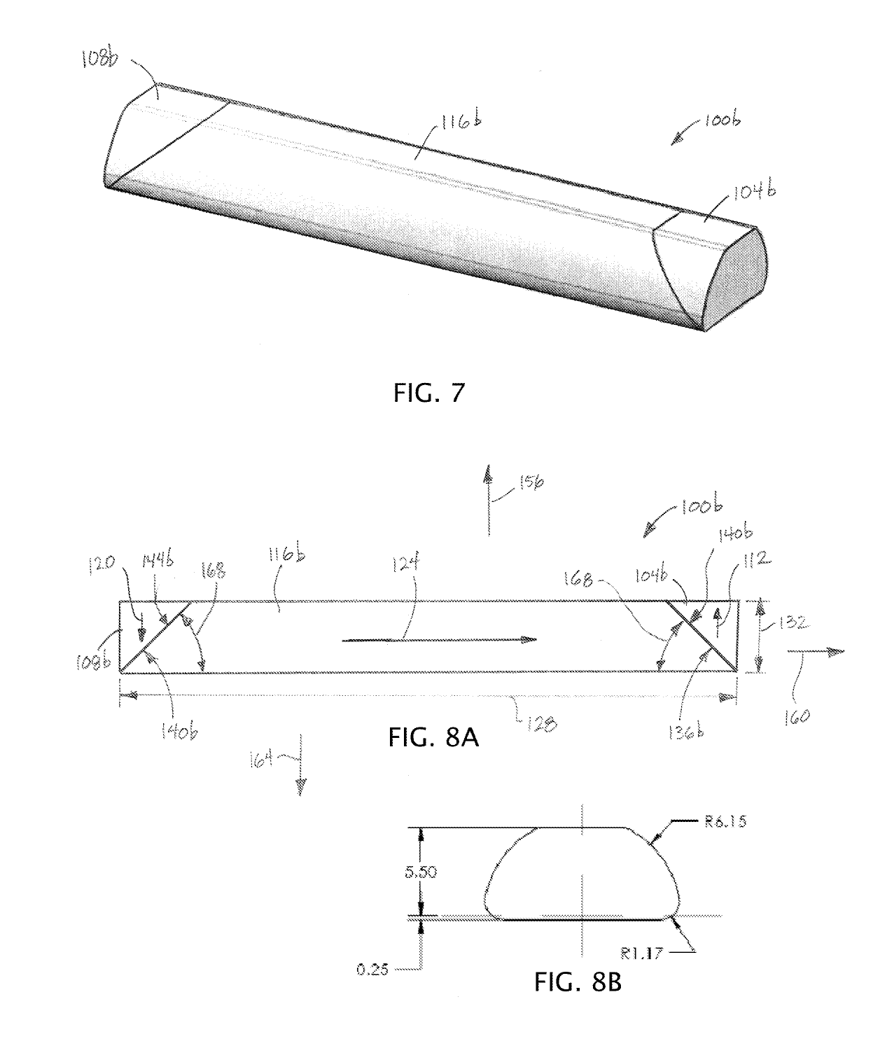

FIG. 7 depicts a perspective view of a second embodiment of three elements for the present medical devices; FIG. 8A depicts a side view of the embodiment of FIG. 7; and FIG. 8B depicts an end view of the embodiment of FIG. 7. In the embodiment shown, a platform or chassis 100b is shown for inclusion in a medical device (e.g., 38) configured to be inserted within a body cavity of a patient. Platform 100b and its components are is similar in some respects to platform 100a and its components. For example, platform 100b includes three or more elements each comprising at least one of a magnetically attractive and magnetically-chargeable material, the three elements at least partially defining a U-shaped magnetic flux path and/or magnetic field. In the embodiment shown, the three or more elements comprise a first element 104b, a second element 108b, and a third element 116b, each of which comprises at least one of a magnetically-attractive material and magnetically-chargeable material. In this embodiment, first element 104b has a first magnetic orientation in which the first element is magnetized and/or magnetizable in a first direction 112; and second element 108b has a second magnetic orientation in which the second element is magnetized and/or magnetizable in a direction 120 that is substantially opposite direction 112. In the embodiment shown, second element 108b is spaced apart from first element 104b, and third element 116b extends between the first element and the second element.

In the embodiment shown, first and second elements 104b and 108b each comprises a magnet magnetized in a N-S direction 112 or 120, respectively. In other embodiments, each of the first and second elements can include a plurality of magnets. In the embodiment shown, third element 116b comprises a magnet and has a magnetic orientation in which the third element is magnetized in direction 124 that is substantially perpendicular to both of directions 112 and 120. In other embodiments, third element 116b can comprise multiple magnets (or pieces of other material), and/or can comprise a ferrous material (e.g., steel such as, for example, a mild steel) that need not be magnetized prior to being in proximity to the first and second elements. In the embodiment shown, third element 116b has an elongated shape in which a length 128 of the third element is larger (e.g., equal to, less than, or between any of: 200%, 500%, 1000%, or more) than a height or thickness 132 of the third element. In the embodiment shown, first, second, and third elements 104b, 108b, 116b are configured such that if coupled together, platform 100b has a substantially constant cross-sectional shape along a length of the platform (along all of the first, second, and third elements), which is equal to length 128 of the third element in the embodiment shown.

In the embodiment shown, mating surfaces 136b and 140b of first and second elements 104b and 108b, respectively, contact or mate with mating surfaces 144b at each end of the third element. In the embodiment shown, mating surfaces 144b (and 136b and 140b) are disposed at a non-perpendicular angle 168 relative to the longitudinal axis (and the bottom surface of) the third element. Angle 168 can be, for example, between 15 and 75 degrees, between 30 and 60 degrees, between 40 and 50 degrees, and/or substantially equal to 45 degrees (as shown). In other embodiments, angle 168 can be varied to maximize attractive force (e.g., in upward direction 156) to an apparatus (e.g., 34), while minimizing unwanted magnetic field projections (e.g., in outward direction 160 and downward direction 164). In the embodiment shown, first, second, and third elements 104b, 108b, and 116c are self-assembling (i.e., the magnet attraction between first and second elements 104b and 108b attract mating surfaces 136b and 144b together, and the magnetic attraction between second and third elements 108b and 116b attract mating surfaces 144b and 140b together. In the embodiment shown, the inclusion of third element 116b increases the magnetic force in upward direction 156, and reduces the overall magnetic field projection in outward direction 160 and downward direction 164, relative to a configuration (FIG. 4) without the third element (with only the first and second elements). FIG. 8B includes one example of dimensions in millimeters, that may also be used in the embodiments of FIGS. 4-6.

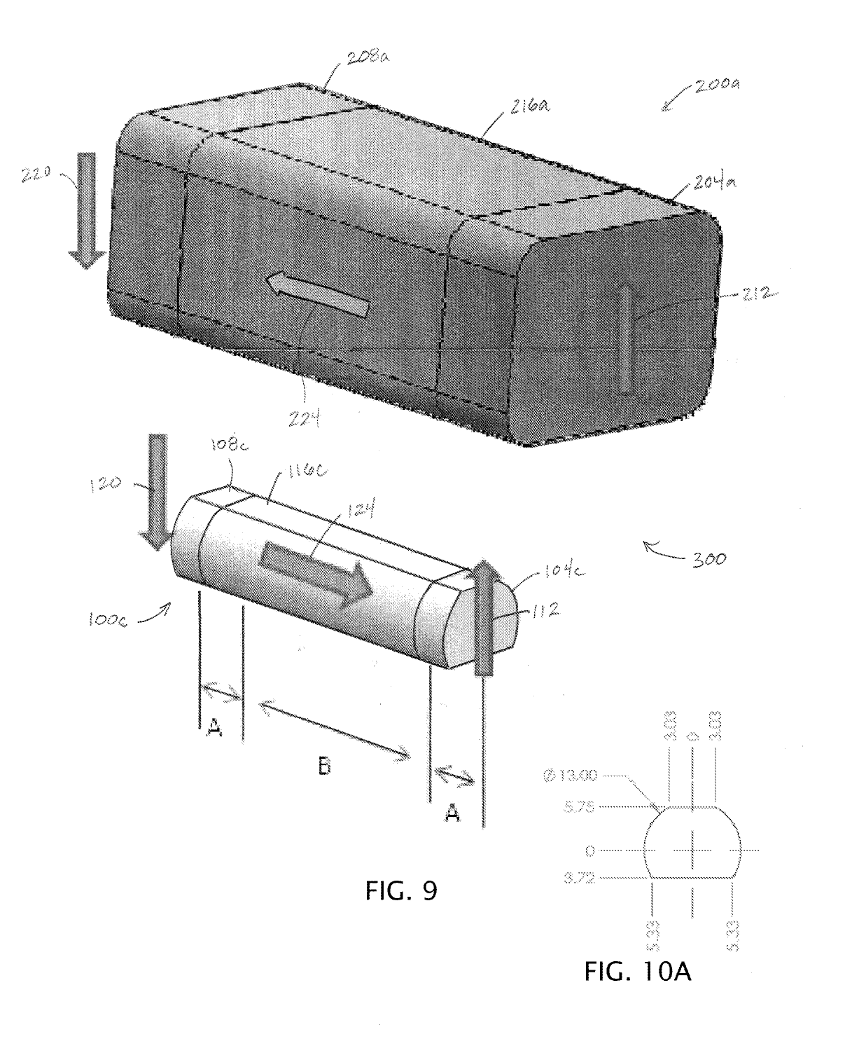

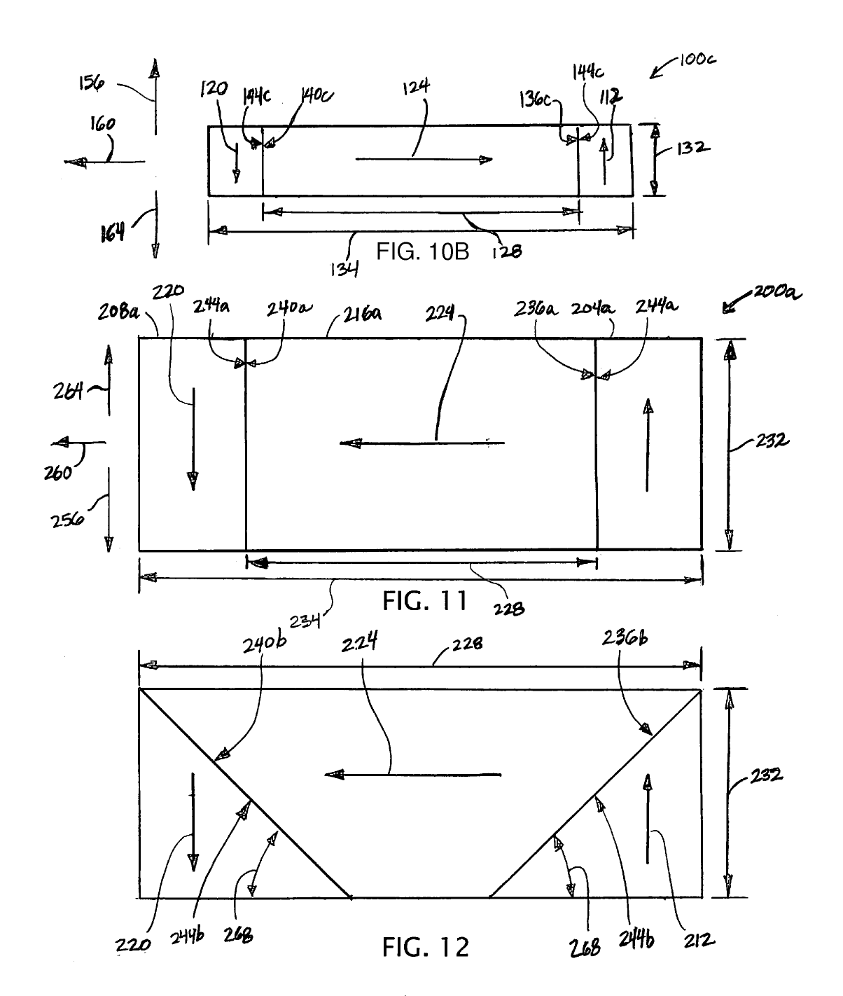

Referring now to FIGS. 9-11, FIG. 9 depicts a perspective view of an embodiment 300 of the present systems that includes a third embodiment of three elements for the present medical devices (e.g., 38) magnetically coupled to an embodiment of three elements for the present apparatuses (e.g., 34); FIG. 10A depicts an end view of the medical-device embodiment of FIG. 9; FIG. 10B depicts a side view of the medical-device embodiment of FIG. 9; and FIG. 11 depicts a side view of the apparatus embodiment of FIG. 9.

The embodiment of FIGS. 10A and 10B is similar in some respects to the embodiment of FIGS. 7 and 8. In the embodiment shown, a platform or chassis 100c is shown for inclusion in a medical device (e.g., 38) configured to be inserted within a body cavity of a patient. Platform 100c and its components are similar in some respects to platform 100b and its components. For example, platform 100c includes three or more elements each comprising at least one of a magnetically attractive and magnetically-chargeable material, the three elements at least partially defining a U-shaped magnetic flux path and/or magnetic field. In the embodiment shown, the three or more elements comprise a first element 104c, a second element 108c, and a third element 116c, each of which comprises at least one of a magnetically-attractive material and magnetically-chargeable material. In this embodiment, first element 104c has a first magnetic orientation in which the first element is magnetized and/or magnetizable in a first direction 112; and second element 108c has a second magnetic orientation in which the second element is magnetized and/or magnetizable in a direction 120 that is substantially opposite direction 112. In the embodiment shown, second element 108c is spaced apart from first element 104c, and third element 116c extends between the first element and the second element.

In the embodiment shown, first and second elements 104c and 108c each comprises a magnet magnetized in a N-S direction 112 or 120, respectively. In other embodiments, each of the first and second elements can include a plurality of magnets. In the embodiment shown, third element 116c comprises a magnet and has a magnetic orientation in which the third element is magnetized in direction 124 that is substantially perpendicular to both of directions 112 and 120. In other embodiments, third element 116c can comprise multiple magnets (or pieces of other material), and/or can comprise a ferrous material (e.g., steel such as, for example, a mild steel) that need not be magnetized prior to being in proximity to the first and second elements. In the embodiment shown, third element 116c has an elongated shape in which a length 128 of the third element is larger (e.g., equal to, less than, or between any of: 200%, 500%, 1000%, or more) than a height or thickness 132 of the third element. In the embodiment shown, first, second, and third elements 104c, 108c, 116c are configured such that if coupled together (as shown), platform 100c has a substantially constant cross-sectional shape along a length 134 of the platform (along all of the first, second, and third elements). In this embodiment, first, second, and third elements 104c, 108c, and 116c each has a substantially identical cross-sectional shape. As shown, external (apparatus) platform 200a is relatively larger than corresponding internal (medical device) platform 100c.

In the embodiment shown, mating surfaces 136c and 140c of first and second elements 104c and 108c, respectively, contact or mate with mating surfaces 144c at each end of the third element. In the embodiment shown, mating surfaces 144c (and 136c and 140c) are substantially perpendicular angle to the longitudinal axis (and the bottom surface of) the third element. In the embodiment shown, the substantially-vertical mating surfaces leverage opposing-pole effects to amplify the magnetic field and the force generated between the apparatus and a magnetically coupled medical device. In this embodiment, the first, second, and third elements are not self-assembling (the magnetic poles of the elements are not arranged to attract the elements together in the configuration shown. For example, the effect of the depicted vertical mating surfaces and magnetization directions is that each of the N-pole and the S-pole of third element 116c equally abuts the N-pole and the S-pole of the respective first or second element 104c or 108c, resulting a state of pure torque on the respective elements at the mating surface, as their respective magnetic fields attempt to turn in order to align the opposing magnetic pole. This stressed state creates a localized, high-intensity field at the interface. As such, force must be applied to assemble the elements as shown (to overcome the magnetic repulsion between the respective elements).

Once assembled in the depicted configuration, the elements must be held together by one or more structures or arrangements (e.g., adhesive, enclosures, etc.). The depicted vertical mating surfaces can result in increased coupling force in direction 156, but may also result in less-smooth transitions in magnetic field between the elements (relative to the configuration of platform 100b with angled mating surfaces) and/or higher peripheral magnetic fields (e.g., in directions 160 and 164). The axial length A of the first and second elements 104c and 108c can be varied relative to the axial length B of third element 116c (e.g., relative to a similarly-configured external apparatus (FIG. 11)) to adjust a force-distance profile (e.g., a force-distance profile in which the curve is “flattened” such that relatively low forces are produced at shorter coupling distances, such as, for example, coupling distances that are less than the thickness of an abdominal wall). In other embodiments, first and second elements 104c and 108c can have magnetic orientations in which the elements are both magnetized horizontally in direction 124, such that opposing magnetic poles are adjacent at the mating surfaces (to make the platform self-assembling). FIG. 10A includes one example of dimensions in millimeters.

In the embodiment of FIG. 11, a platform 200a is shown for inclusion in an apparatus (e.g., 34) such as an external control apparatus or unit (ECU) that is configured to be magnetically coupled to a medical device (eg., 38). Platform 200a and its components are similar in some respects to platform 100c and its components. For example, platform 200c includes three or more elements each comprising at least one of a magnetically attractive and magnetically-chargeable material, the three elements at least partially defining a U-shaped magnetic flux path and/or magnetic field. In the embodiment shown, the three or more elements comprise a first element 204a, a second element 208a, and a third element 216a, each of which comprises at least one of a magnetically-attractive material and magnetically-chargeable material. In this embodiment, first element 204a has a first magnetic orientation in which the first element is magnetized and/or magnetizable in a first direction 212; and second element 208a has a second magnetic orientation in which the second element is magnetized and/or magnetizable in a direction 220 that is substantially opposite direction 212. In the embodiment shown, second element 208a is spaced apart from first element 204a, and third element 216a extends between the first element and the second element. First and second elements 204a and 208a, for example, can be similar in materials and/or function to magnetic field sources 62a and 62b, described above.

In the embodiment shown, first and second elements 204a and 208a each comprises a magnet magnetized in a N-S direction 212 or 220, respectively. In other embodiments, each of the first and second elements can include a plurality of magnets. In the embodiment shown, third element 216c comprises a magnet and has a magnetic orientation in which the third element is magnetized in direction 224 that is substantially perpendicular to both of directions 212 and 220. In other embodiments, third element 216a can comprise multiple magnets (or pieces of other material), and/or can comprise a ferrous material (e.g., steel such as, for example, a mild steel) that need not be magnetized prior to being in proximity to the first and second elements. In the embodiment shown, third element 216c has an elongated shape in which a length 228 of the third element is larger (e.g., equal to, less than, or between any of: 150%, 200%, 300%, 500%, 1000%, or more) than a height or thickness 232 of the third element. In the embodiment shown, first, second, and third elements 204a, 208a, 216a are configured such that if coupled together (as shown), platform 200a has a substantially constant cross-sectional shape along a length 234 of the platform (along all of the first, second, and third elements). In this embodiment, first, second, and third elements 204a, 208a, and 216a each has a substantially identical cross-sectional shape.

In the embodiment shown, mating surfaces 236a and 240a of first and second elements 204a and 208a, respectively, contact or mate with mating surfaces 244a at each end of the third element. In the embodiment shown, mating surfaces 244a (and 236a and 240a) are substantially perpendicular angle to the longitudinal axis (and the bottom surface of) the third element. In the embodiment shown, the substantially-vertical mating surfaces leverage opposing-pole effects to amplify the magnetic field and the force generated between the apparatus and a magnetically coupled medical device. In this embodiment, the first, second, and third elements are not self-assembling (the magnetic poles of the elements are not arranged to attract the elements together in the configuration shown. For example, the effect of the depicted vertical mating surfaces and magnetization directions is that each of the N-pole and the S-pole of third element 216a equally abuts the N-pole and the S-pole of the respective first or second element 204a or 208a, resulting a state of pure torque on the respective elements at the mating surface, as their respective magnetic fields attempt to turn in order to align the opposing magnetic pole. This stressed state creates a localized, high-intensity field at the interface. As such, force must be applied to assemble the elements as shown (to overcome the magnetic repulsion between the respective elements).

Once assembled in the depicted configuration, the elements must be held together by one or more structures or arrangements (e.g., adhesive, enclosures, etc.). The depicted vertical mating surfaces can result in increased coupling force in direction 256, but may also result in less-smooth transitions in magnetic field between the elements (e.g., relative to angled mating surfaces (FIG. 12)) and/or higher peripheral magnetic fields (e.g., in directions 260 and 264). The axial length A of the first and second elements 204a and 208a can be varied relative to the axial length B of third element 216a (e.g., relative to a similarly-configured medical device (FIG. 10)) to adjust a force-distance profile (e.g., a force-distance profile in which the curve is “flattened” such that relatively low forces are produced at shorter coupling distances, such as, for example, coupling distances that are less than the thickness of an abdominal wall). In other embodiments, first and second elements 204a and 208a can have magnetic orientations in which the first and second elements are both magnetized horizontally in direction 224, such that opposing magnetic poles are adjacent at the mating surfaces (to make the platform self-assembling).

FIG. 12 depicts a side view of a second embodiment of three elements for the present apparatuses. In the embodiment shown, a platform 200b is shown for inclusion in an apparatus (e.g., 34) such as an external control apparatus or unit (ECU) that is configured to be magnetically coupled to a medical device (eg., 38). Platform 200b and its components are similar in some respects to platform 200a and its components. For example, platform 200b includes three or more elements each comprising at least one of a magnetically attractive and magnetically-chargeable material, the three elements at least partially defining a U-shaped magnetic flux path and/or magnetic field. In the embodiment shown, the three or more elements comprise a first element 204b, a second element 208b, and a third element 216b, each of which comprises at least one of a magnetically-attractive material and magnetically-chargeable material. In this embodiment, first element 204b has a first magnetic orientation in which the first element is magnetized and/or magnetizable in a first direction 212; and second element 208b has a second magnetic orientation in which the second element is magnetized and/or magnetizable in a direction 220 that is substantially opposite direction 212. In the embodiment shown, second element 208b is spaced apart from first element 204b, and third element 216b extends between the first element and the second element.

In the embodiment shown, first and second elements 204b and 208b each comprises a magnet magnetized in a N-S direction 212 or 220, respectively. In other embodiments, each of the first and second elements can include a plurality of magnets. In the embodiment shown, third element 216b comprises a magnet and has a magnetic orientation in which the third element is magnetized in direction 224 that is substantially perpendicular to both of directions 212 and 220. In other embodiments, third element 216b can comprise multiple magnets (or pieces of other material), and/or can comprise a ferrous material (e.g., steel such as, for example, a mild steel) that need not be magnetized prior to being in proximity to the first and second elements. In the embodiment shown, third element 216b has an elongated shape in which a length 228 of the third element is larger (e.g., equal to, less than, or between any of: 150%, 200%, 300%, 500%, 1000%, or more) than a height or thickness 232 of the third element. In the embodiment shown, first, second, and third elements 204b, 208b, 216b are configured such that if coupled together (as shown), platform 200b has a substantially constant cross-sectional shape along a length of the platform (along all of the first, second, and third elements), which is equal to length 228 of the third element, in the embodiment shown.

In the embodiment shown, mating surfaces 236b and 240b of first and second elements 204b and 208b, respectively, contact or mate with mating surfaces 244b at each end of the third element. In the embodiment shown, mating surfaces 244b (and 236b and 240b) are disposed at a non-perpendicular angle 268 relative to the longitudinal axis (and the bottom surface of) the third element. Angle 268 can be, for example, between 15 and 75 degrees, between 30 and 60 degrees, between 40 and 50 degrees, and/or substantially equal to 45 degrees (as shown). In other embodiments, angle 268 can be varied to maximize attractive force (e.g., in downward direction 256) to a medical device (e.g., 34), while minimizing unwanted magnetic field projections (e.g., in outward direction 260 and upward direction 264). In the embodiment shown, first, second, and third elements 204b, 208b, and 216c are self-assembling (i.e., the magnet attraction between first and second elements 204b and 208b attract mating surfaces 236b and 244b together, and the magnetic attraction between second and third elements 208b and 216b attract mating surfaces 244b and 240b together. In the embodiment shown, the inclusion of third element 216b increases the magnetic force in downward direction 256, and reduces the overall magnetic field projection in outward direction 260 and upward direction 264, relative to a configuration without the third element (with only the first and second elements).

Any of the present ECU or external platforms 200a, 200b can be used (magnetically coupled) with any of the medical device or internal platforms 100a, 100b, 100c. For example, platform 200b can be used with platform 100c.

The above specification and examples provide a complete description of the structure and use of exemplary embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the present devices are not intended to be limited to the particular forms disclosed. Rather, they include all modifications and alternatives falling within the scope of the claims, and embodiments other than the one shown may include some or all of the features of the depicted embodiment. For example, components may be combined as a unitary structure, and/or connections may be substituted (e.g., threads may be substituted with press-fittings or welds). Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A medical device comprising:
a platform configured to be inserted within a body cavity of a patient, the platform comprising:
three or more elements each comprising at least one of a magnetically attractive and magnetically-chargeable material, the three elements at least partially defining a U-shaped magnetic flux path.

2. The medical device of claim 1, where the one or more elements comprise:
a first element comprising at least one of a magnetically-attractive material and magnetically-chargeable material, the first element having a first magnetic orientation;
a second element comprising at least one of a magnetically-attractive material and magnetically-chargeable material, the second element having a second magnetic orientation; and
a third element comprising at least one of a magnetically-attractive material and magnetically-chargeable material;
where the second element is spaced apart from the first element, the second magnetic orientation is opposite the first magnetic orientation, and the third element extends between the first element and the second element.

3. The medical device of claim 2, where the third element has a third magnetic orientation independent of the first and second elements.

4. The medical device of claim 3, where the third magnetic orientation is substantially perpendicular to the first and second magnetic orientations.

5. The medical device of claim 2, where the third element has an elongated shape and a central longitudinal axis.

6. The medical device of claim 5, where the third element has a first mating surface at a first end, and a second mating surface at a second end.

7. The medical device of claim 6, where the first and second mating surfaces of the third element are substantially perpendicular to the longitudinal axis.

8.-9. (canceled)

10. The medical device of claim 1, where the first and second elements have substantially identical cross-sectional shapes.

11. The medical device of claim 1, where the first, second, and third elements have substantially identical cross-sectional shapes.

12. An apparatus comprising:
a platform configured to be magnetically coupled to a medical device disposed within a body cavity of a patient through a tissue, the platform comprising:
a body; and
three elements each comprising at least one of a magnetically attractive and magnetically-chargeable material, the three elements at least partially defining a U-shaped magnetic flux path.

13. The apparatus of claim 13, where the one or more elements comprise:
a first element comprising at least one of a magnetically-attractive material and magnetically-chargeable material, the first element having a first magnetic orientation;
a second element comprising at least one of a magnetically-attractive material and magnetically-chargeable material, the second element having a second magnetic orientation; and
a third element comprising at least one of a magnetically-attractive material and magnetically-chargeable material;
where the second element is spaced apart from the first element, the second magnetic orientation is opposite the first magnetic orientation, and the third element extends between the first element and the second element.

14. The apparatus of claim 13, where the third element has a third magnetic orientation independent of the first and second elements.

15. The apparatus of claim 14, where the third magnetic orientation is substantially perpendicular to the first and second magnetic orientations.

16. The apparatus of claim 13, where the third element has an elongated shape and a central longitudinal axis.

17. The apparatus of claim 16, where the third element has a first mating surface at a first end, and a second mating surface at a second end.

18. The apparatus of claim 17, where the first and second mating surfaces of the third element are substantially perpendicular to the longitudinal axis.

19.-20. (canceled)

21. The apparatus of claim 12, where the first and second elements have substantially identical cross-sectional shapes.

22. The apparatus of claim 13, where the first, second, and third elements have substantially identical cross-sectional shapes.

23. A system comprising:
an apparatus of claim 12;
a medical device configured to be inserted within a body cavity of a patient, the medical device comprising:
a platform comprising one or more elements having at least one of a magnetically attractive and magnetically-chargeable material.

24. The system of claim 23, where the one or more elements of the medical device at least partially define a U-shaped magnetic flux path.

25. The system of claim 24, where the one or more elements of the medical device comprise:
a first element comprising at least one of a magnetically-attractive material and magnetically-chargeable material, the first element having a first magnetic orientation;
a second element comprising at least one of a magnetically-attractive material and magnetically-chargeable material, the second element having a second magnetic orientation; and
a third element comprising at least one of a magnetically-attractive material and magnetically-chargeable material;
where the second element is spaced apart from the first element, the second magnetic orientation is opposite the first magnetic orientation, and the third element extends between the first element and the second element.

26. The system of claim 23, where the apparatus is magnetically coupled to the medical device.

27. A system comprising:
an apparatus configured to be coupled to a medical device within a body cavity of a patient;
a medical device of claim 1.

28. The system of claim 27, where the apparatus comprises an apparatus of claim 12.

29. The system of claim 27, where the apparatus is magnetically coupled to the medical device.
